(19) United States
(12) Patent Application Publication
(10) Pub. No.: US 2016/0244709 A1
(43) Pub. Date: Aug. 25, 2016

(54) Patterned Magnetic Thin Film with Rolled-Up Hollow Structure

(71) Applicant: National Tsing Hua University, Hsin Chu City (TW)

(72) Inventors: Zung-Hang WEI, Hsin Chu City (TW); Chen-Yu HUANG, Hsin Chu City (TW)

(21) Appl. No.: 14/753,018

(22) Filed: Jun. 29, 2015

(30) Foreign Application Priority Data
Feb. 24, 2015 (TW) 104105891

(51) Int. Cl. C12M 1/12; C12M 1/42

(52) U.S. Cl. CPC C12M 25/10; C12M 35/06

(57) ABSTRACT

The present invention provides a patterned magnetic thin film with 3D tube-shaped structure. The magnetic thin film includes a substrate which includes at least one tube-shaped supportive layer. The tube-shaped supportive layer is rolled up onto the substrate. And the tube-shaped supportive layer further includes a pattern portion which having one or more magnetic material to attract an object into a hollow portion of the tube-shaped supportive layer.

### CROSS-REFERENCE TO RELATED APPLICATIONS

This application claim priority to TAIWAN application Numbered 104105891, filed Feb. 24, 2015, which is herein incorporated by reference in its' integrity.

### TECHNICAL FIELD

The present invention generally relates to a patterned magnetic thin film, and more particularly, to an integration of patterned magnetic thin film with rolled-up hollow structure.

### BACKGROUND OF RELATED ART

Cancer seriously affects human being. Cancer is a class of diseases which occurs as cell become immortalized. Most early symptoms of cancer are unable to be observed easily. But in terminal cancer, cancer cells may spread out to other parts through blood. It is worth to develop a new treatment to detect cancer cell early so as to inhibit it degradation.

Animals are formed by numerous and tiny units, such cell, vessel, and so on. In recent, scientists try to creating devices using microelectromechanical technology for in vitro applications, such as cell culture, and detecting and sensing thereof, to realize the in vivo environment.

The benefit of applying magnetic film for biologic application is to reduce cell damage during the collection/detection process, furthermore, improve drug test on particular cell and/or cell cluster. In prior art, most magnetic films are restricted to 2D planar structures which can be modified by functional group or defined with specific patterns thereon, for increasing collection and detection. However, the amount of collected cells are limited by planar space and magnetic property of cells. Furthermore, 2D planar magnetic thin film is not useful for separating particular cells from cell cluster.

In prior art, it has developed thin film with three-dimensional (3D) structure to improve the efficiency of detection, but in conventional manufacturing process, 3D structure must firstly be prepared on the substrate, and then followed by other material covered onto the 3D structure; therefore, it is complicated to prepare 3D magnetic thin film by conventional process.

In order to solve the problem of the conventional arts, there is a need to provide simple apparatus for improving detection and preparing. The present invention remains normal 2D characteristics but improves detection property by forming 3D structure.

### SUMMARY

An object of the present invention is to provide a patterned thin film with tube-shaped structure to generate particular stray field and collect large amount of cells.

Another object of the present invention is to provide a method for preparing a patterned thin film with tube-shaped structure. The rolled-up structure was then obtained due to the different thermal expansion coefficient of material.

According to one embodiment, the patterned thin film with tube-shaped structure includes at least one substrate. The substrate includes at least one tube-shaped supportive layer rolled up onto the substrate. The tube-shaped thin film further includes a pattern portion having magnetic material for attracting an object into the hollow portion of the tube-shaped thin film. Wherein the thermal expansion coefficient of the magnetic material and the tube-shaped supportive layer are different.

According to one embodiment, the method of preparing the patterned thin film with tube-shaped structure includes following steps: preparing a substrate; covering a supportive layer onto the substrate; defining a pattern portion onto the supportive layer; depositing magnetic material onto the pattern portion; opening a concavity on at a side of the supportive layer; and removing the substrate by etching.

### BRIEF DESCRIPTION OF THE DRAWINGS

The components, characteristics and advantages of the present invention may be understood by the detailed description of the preferred embodiments outlined in the specification and the drawings attached.

FIG. 1 illustrates a flow chart of preparing the patterned magnetic thin film with 3D tube-shaped structure according to an embodiment of the present invention.

FIG. 2A illustrates a sectional view of the substrate, supportive layer and magnetic material according to an embodiment of the present invention.

FIG. 2B illustrates a sectional view of opening a concavity at a side of the supportive layer according to an embodiment of the present invention.

FIG. 2C illustrates a sectional view of rolled-up structure according to an embodiment of the present invention.

FIG. 3A illustrates a diagram of supportive layer without pattern according to an embodiment of the present invention.

FIG. 3B illustrates a diagram of the supportive layer with pattern according to an embodiment of the present invention.

FIG. 3C illustrates a diagram of concavities at three sides of the supportive layer according to an embodiment of the present invention.

FIG. 3D illustrates a diagram of etching substrate and forming rolled-up structure according to an embodiment of the present invention.

FIG. 4A-4B illustrate diagrams of the patterned magnetic thin film with rolled-up structure according to an embodiment of the present invention.

FIG. 5A illustrates a diagram of the magnetic thin film with single periodic pattern before rolling-up according to an embodiment of the present invention.

FIG. 5B illustrates a diagram of the magnetic thin film with single periodic pattern with rolled-up structure according to an embodiment of the present invention.

FIG. 6A illustrates a diagram of the magnetic thin film with M periodic pattern before rolling-up according to an embodiment of the present invention.

FIG. 6B illustrates a diagram of the magnetic thin film with M periodic pattern with rolled-up structure according to an embodiment of the present invention.

FIG. 7A-7B illustrate diagrams of the patterned magnetic thin film with rolled-up structure according to an embodiment of the present invention.

FIG. 8A illustrates a diagram of the objective cells collected by the magnetic thin film with rolled-up structure according to an embodiment of the present invention.

FIG. 8B illustrates the curve of number of collected cells versus collection time of the tube-shaped with patterned magnetic thin film according to an embodiment of the present invention.

### DETAILED DESCRIPTION

Some preferred embodiments of the present invention will now be described in greater detail. However, it should be recognized that the preferred embodiments of the present invention are provided for illustration rather than limiting the present invention. In addition, the present invention can be practiced in a wide range of other embodiments besides those explicitly described, and the scope of the present invention is not expressly limited except as specified in the accompanying claims. The layout of components may be more complicated in practice.

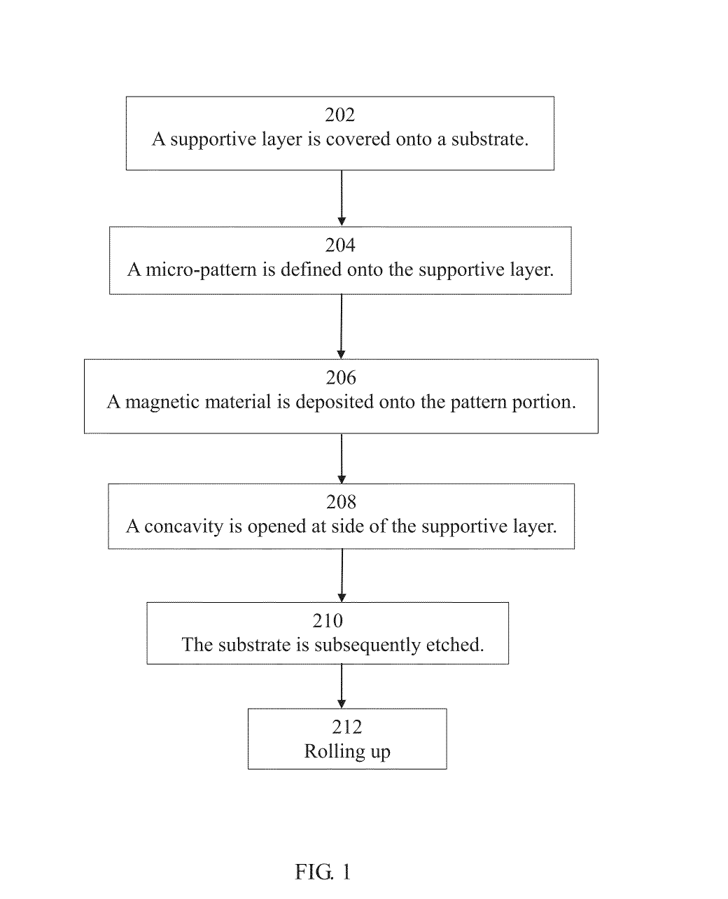

FIG. 1 illustrate a flow chart of preparing a patterned magnetic thin film with rolled-up hollow structure according to an embodiment of the present invention. The method provides at least one substrate, such as silicon material, for following steps:

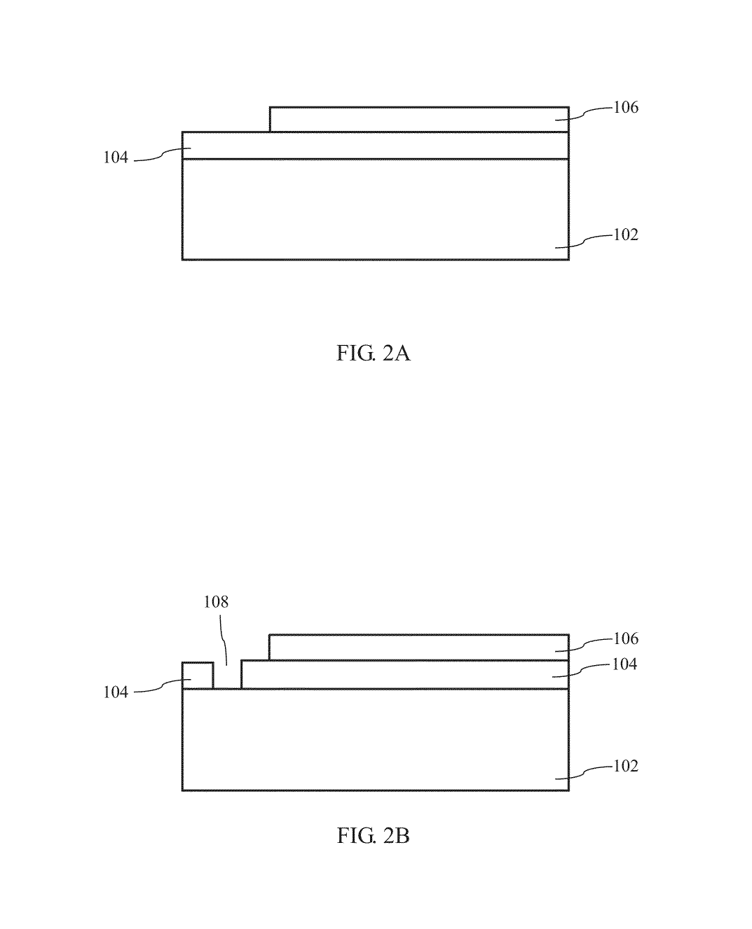

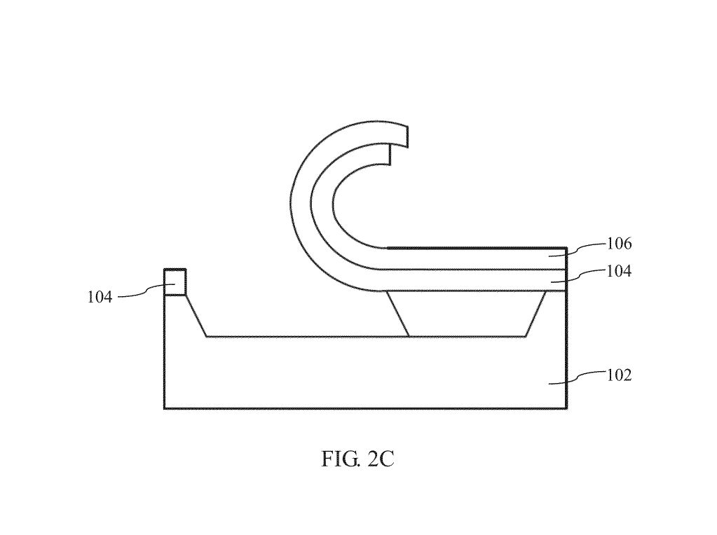

Step 202: A supportive layer 104 is covered over the substrate 102. FIG. 2A illustrates a sectional view of the substrate 102, a supportive layer 104 and a magnetic material 106 of the present invention. The supportive layer 104 includes but is not limited to SiO₂ or Si₃N₄, in the preferred embodiment, the supportive layer 104 is SiO₂. The supportive layer 104 is formed over the substrate 102 by coating, printing or other process. In the preferred embodiment, the supportive layer 104 is covered over the substrate 102 by coating. The thickness of the supportive layer 104 is can be about 10-100 nm, more particularly, about 100 nm.

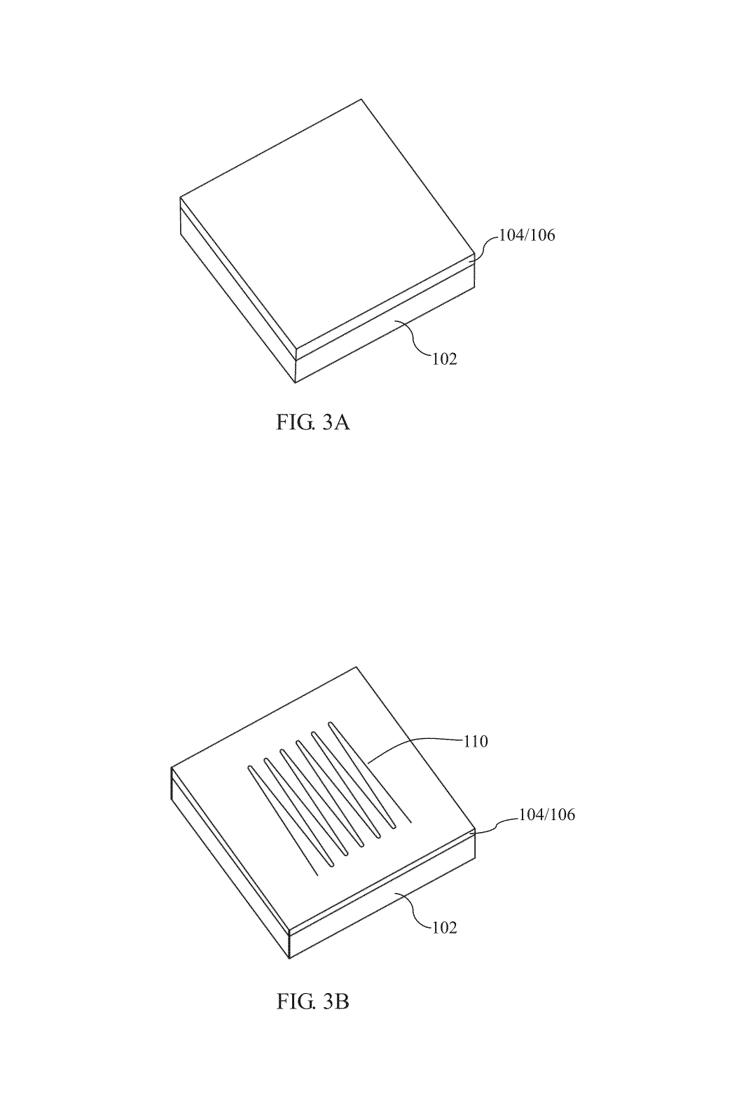

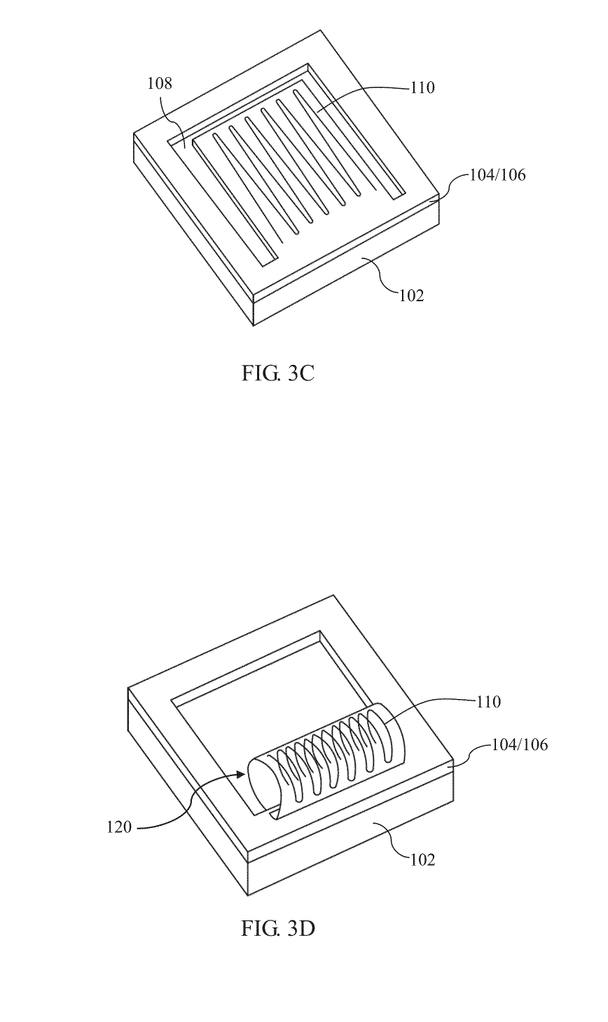

Step 204: A micro-pattern is defined on the supportive layer 104. FIGS. 3A-3D illustrate processes of patterning portion 110 formed on the supportive layer 104. It is well understood that the present invention must coat a photoresist agent on the surface of the supportive layer 104 in order to define a pattern. Either positive resist or negative resist can be adapted for defining patterns based on the specific requirements. In the preferred embodiment, a positive resist polymethylmethacrylate (PMMA) is covered on the supportive layer 104 by spin coating, then continues following steps.

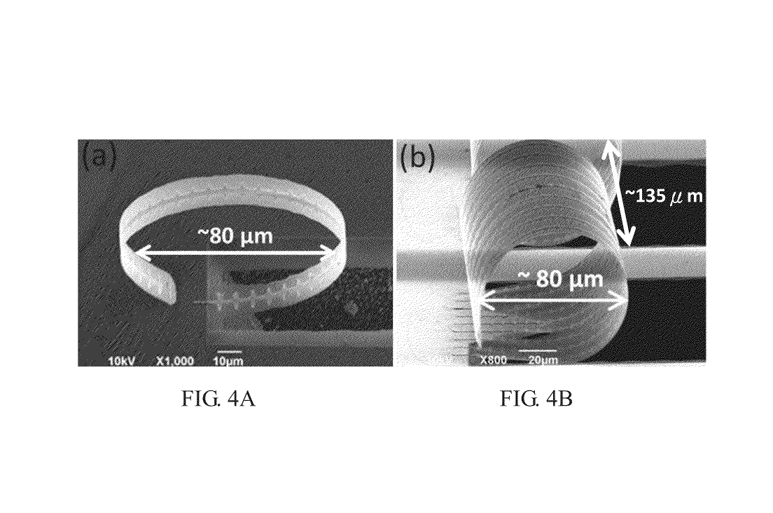

Lithography is an important part in process of semiconductor and micro electro mechanism (MEM), most of the patterned area can be defined by lithography. Lithographic technique includes extreme ultraviolet lithography (EUV), X-ray lithography, electron projection lithography (EPL), ion projection lithography (IPL), and so on. Electron-beam lithography (often abbreviated as e-beam lithography) is the practice of scanning a focused beam of electrons to draw custom shapes on a surface covered with an electron-sensitive film called a resist. The electron beam changes the solubility of the resist, enabling selective removal of either the exposed of non-exposed regions of the resist by immersing it in a solvent. In the preferred embodiment, the pattern portion 110 are created onto the substrate 102 that spin-coated with e-beam resist polymethyl methacrylate (PMMA). Then, the pattern portion 110 will be appeared on the substrate 102 in developer, such as 3:1 mixture of 2-propanol and methyl isobutyl ketone. The pattern portion 110 includes any micro patterns, such as but not limited to linear, wave-shaped (as shown in FIG. 3B), or fishbone-shaped (as shown in FIGS. 4A-4B). It is well understood that the lithographic technique is not limited to e-beam lithography, but can be varied or modified by the person in the art in the light of the need in use. Besides, in step 204, the method further includes dehydration baking, priming, soft baking and hard baking to enhance precision and reliability of the pattern portion 110.

Step 206: A magnetic material 106 is deposited onto the pattern portion 110. A required material can be coated onto the supportive layer 104, such as but not limited to magnetic material, conductive material, non-conductive material or semiconductive material, after defining the pattern portion. In one embodiment, the magnetic material 106, coated onto the surface of the supportive layer 104. In the preferred embodiment, the magnetic material 106 is deposited onto the surface of the supportive layer 104 by e-beam evaporation. In the preferred embodiment, the magnetic material 106 comprises a first layer of non-magnetic material as adhesive layer, a second layer of magnetic layer as sensing layer and a third layer as protective layer. Non-magnetic material includes but not limited to Cr, Ti, Al; magnetic metal includes but not limited to Fe, Co, Ni, or nickel-, iron- and cobalt-based alloys; protective layer includes but not limited to Cr, Ti, Al or polymer (not shown in drawings). We used the e-beam evaporation system to deposit (1) about 5-20 nm thick Cr as the adhesive layer, preferable 10 nm; (2) second layer of Ni₈₀Fe₂₀ ranges from 30 nm to several micrometers as the sensing layer, preferable 90 nm; and (3) about 5-20 nm thick Cr as the protective layer, preferable 10 nm, in sequence. Accordantly, 2D patterned thin film with will be done through above steps. It is well understood that different magnetic material 106 can be deposited with interlaced format and repeatability to modulate the magnetic strength.

In another embodiment, particular functional groups can be modified onto the surface of the supportive layer 104, such as but not limited protein or DNA, for trapping or attracting particular cells.

Step 208: A concavity 108 is opened on at a side of the supportive layer 104. FIG. 2B illustrates a sectional view of the concavity 108 formed at a side of the supportive layer 104. FIG. 3C illustrate a perspective view of the opening 108 formed at a side of the supportive layer 104. First, required concavities are defined onto the supportive layer 104 and the magnetic material 106 by lithography, and then the concavities are etched by buffered oxide etchant (BOE). As shown in FIG. 3C, each of left, right and front side of the supportive layer 104 and the magnetic material 106 has a concavity, respectively, for forming rolled-up thin film 120 (also called tube-shaped thin film or ring-structure thin film). It is well understood that the height and width of concavities 108 can be modified or varied based on the requirements by a skilled person in the art. In the embodiment, the width of the concavity 108 is 50 micrometers. The length of the two concavities 108, formed at the right and left sides of the supportive layer 104 and the magnetic material 106, determines the diameter of the tube-shaped thin film 120. As shown in FIG. 4A, it illustrates a front view of tube-shaped thin film 120 by SEM, the diameter of the tube-shaped thin film is 80 micrometers. The length of the two concavities 108, formed at the front and back sides of the supportive layer 104 and the magnetic material 106, determines the length of the tube-shaped thin film 120. As shown in FIG. 4B, it illustrates a front view of tube-shaped thin film 120 by SEM, the length of the tube-shaped thin film is 135 micrometers.

Step 210: The substrate is subsequently etched. The substrate 102, after step 208, is immersed into etchant, such as tetramethylammonium hydroxide (TMAH) for removing parts of the substrate 102 to form the tube-shaped thin film 120. Referring to FIG. 2C, the supportive layer 104 and the magnetic material 106 bend away or curl towards a side of the substrate 102 in etching process, due to the difference in thermal expansion coefficient between the supportive layer 104 and the magnetic material 106 in etching process. In the embodiment, the etchant includes but is not limited to TMAH (N(CH₃)₄⁺OH⁻).

Step 212: The magnetic material 106 and the supportive layer 104 can roll up owing to stress induced by the difference in thermal expansion between different layers being released after substrate etched. If the thermal expansion coefficient of the supportive layer 104 is greater than that of the magnetic material 106, they will bent towards a side of the supportive layer 104 (away from a side of the magnetic material 106), thereby rolling downward (not shown in figures.) If the thermal expansion coefficient of the supportive layer 104 is smaller than the magnetic material 106, they will bent towards a side of the magnetic material 106 (away from a side of the supportive layer 104), thereby rolling upward and forming the tube-shaped thin film 120, as shown in FIGS. 2C and 3D. In the preferred embodiment, the thermal expansion coefficient of Cr, Ni₈₀Fe₂₀ and SiO₂ are 6.2 (10⁻⁶/mK), 12.8 (10⁻⁶/mK) and 0.5 (10⁻⁶/mK), respectively, so the supportive layer 104 will bent towards a side of Ni₈₀Fe₂₀. The difference of thermal expansion coefficient between the magnetic 106 and the supportive layer 104 is about 4.8-12.3 (10⁻⁶/mK). Furthermore, the present invention can develop tube-shaped structure with multiple thin film. Beside, multiple 3D tube-shaped thin films can be prepared onto the same substrate.

On the other hand, diameter and turns can be modulated by external factors, such as etching time and temperature. In one embodiment, etching rate rises as temperature from 60° C. to 150° C., and thus the number of turns (N) of the tube-shaped thin film 120 will be made. In one embodiment, the number of turns (N) is 3 under temperature between 90° C.-110° C.; in contrary, the number of turns (N) is 1 under temperature between 60° C.-80° C. Accordantly, the number of turns (N) are proportional to the temperature. It is well understood that the desired operating temperature is based on the depositing material chosen in thin film material layer.

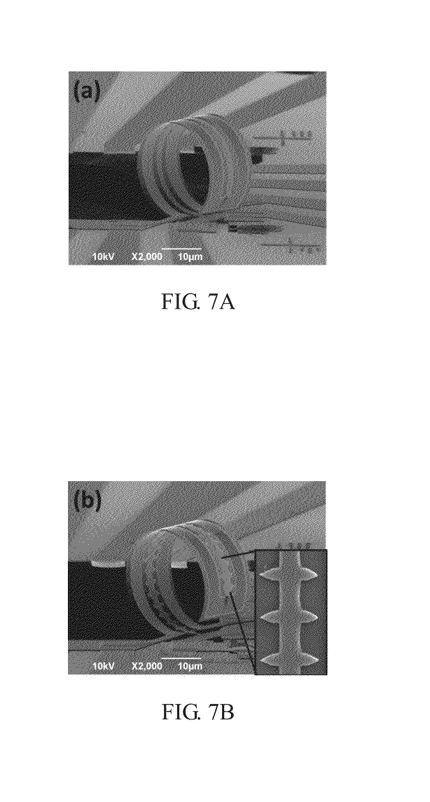

The pattern portion 110 defined onto the supportive layer 104 includes various type, such as simple linear or wave-shaped. In one embodiment, another type, such as elliptical or oval shape with width-to-length ratio being 1:2-1:10, are perpendicular to the simple linear or wave-shaped type to form particular type with high magnetic anisotropy, such as fishbone-shaped type, to enhance the sensitivity of magnetic detection. Type of the pattern portion 110 can be arranged regularly or randomly, type and its arrangement of the pattern portion 110 can be modified or varied based on the requirements, as shown in FIG. 7A-7B. FIG. 7A illustrates the simple linear type, and the FIG. 7B illustrates the elliptical type, with width-to-length ratio 1:5, being perpendicular to the simple linear type.

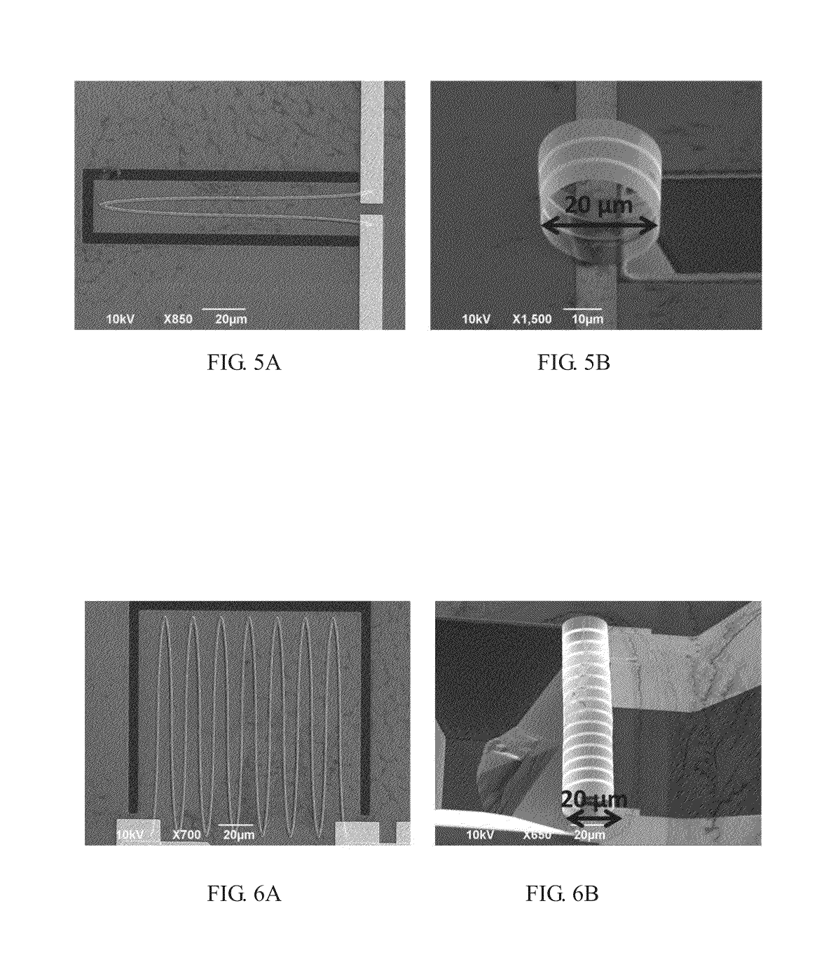

FIG. 5A illustrates a magnetic thin film with single periodic type before rolling up. FIG. 6A illustrates a magnetic thin film with seven periodic type before rolling up. The pattern portion 110 of the supportive layer 104 includes M periodic type, as shown in FIGS. 3C, 5A and 6A. After depositing the magnetic material 106 onto the pattern portion 110 of the supportive layer 104, the tube-shaped thin film 120 will be made through heating and etching, thereby enhancing magnetic collection and sensitivity thereof, as shown in FIGS. 3D, 5B and 6B. FIG. 5B illustrates a side views of tube-shaped thin film with single periodic type, FIG. 6B illustrates a side view of tube-shaped thin film with multiple periodic patterns.

In one embodiment, the magnetic material includes but is not limited to Cr coated on Ni₈₀Fe₂₀. Magnetic objects, such as magnetic cell, magnetic molecule and magnetic bead, can be collected and attracted by the stray field of the 3D magnetic film 120 due to the patterned magnetic film 120 has particular magnetic anisotropy.

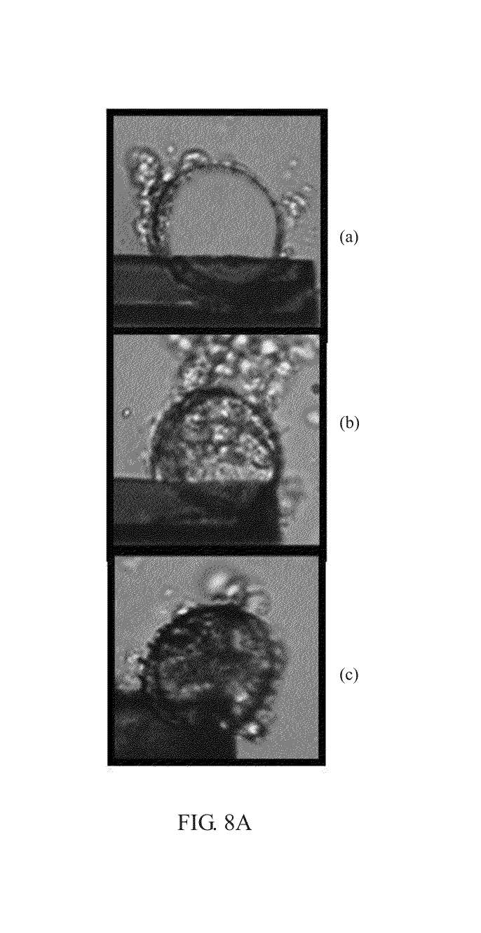

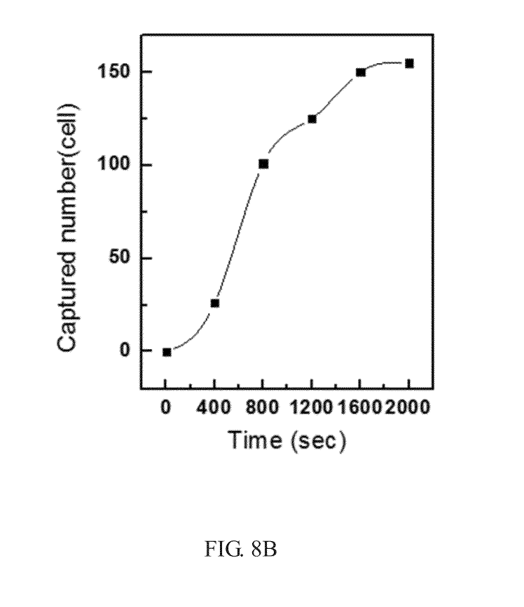

The number of the magnetic objects attracted are depended on the diameter and length of the tube-shaped thin film 120. In one embodiment, magnetically labeled cancer cells are collected into the hollow portion of tube-shaped thin film 120, the diameter of the hollow portion is 60 micrometers. As shown in FIG. 8A, (a), (b) and (c) illustrate diagrams of magnetic cells collected by the tube-shaped thin film 120 after 200, 1000 and 1800 seconds, respectively. FIG. 8B illustrates the curve of number of collected cells versus collection time, the maximum number of collected cells in FIG. 8B is about 150. In another embodiment, the similar size and amount of cell cluster can be collected into the tube-shaped thin film 120 by modulating the size of diameter of the tube-shaped thin film 120, for analysis.

Rolled-up structure can be as a scaffold for 3D cell culture. On the other hand, the rolled-up thin film can capture a particular cell from cell cluster.

Table 1 presents the switching field variation of 2D magnetic thin film and 3D magnetic thin film trapping a magnetic cell. The Hc₁ indicates coercivity of a cell attached, the Hc₀ indicates coercivity of no cell attached, wherein Hc₁ minus Hc₀ equals the switching field variation. Long and short axes in Table 1 respectively indicate the magnetic field applied along the long axis or short axis of device. According to the Table 1, the switching field variation of 3D magnetic thin film are greater than those of 2D magnetic thin film as magnetic field applied in either axis, in other words, the variation of magnetic signal of 3D thin film are greater than those of 2D thin film, and thus 3D magnetic thin film is better to as magnetic sensor or biosensor. That is, rolled-up magnetic thin film can be served as biosensor to enhance signal and sensitivity, and reduce deviation.

| switching field | 2D sensor | 3D sensor |
|---|---|---|
| variation (%) | with a cell | with a cell |
| Long axis | 12 | 62.5 |
| Short axis | 20.2 | 41.4 |

As description above, the present invention provides a patterned magnetic thin film with 3D rolled-up structure. The 3D rolled-up thin film can be served as biosensor to dissolve disadvantage of conventional 2D sensor due to its magnetic thin film having patterns with high magnetic anisotropy. In addition, the 3D rolled-up thin film also increase the amount of collected cells and detective direction as a result of its rolled-up structure which can enhance the signal.

Various terms used in this disclosure should be construed broadly. For example, if an element “A” is said to be coupled to or with element “B,” element A may be directly coupled to element B or be indirectly coupled through, for example, element C. When the specification states that a component, feature, structure, process, or characteristic A “causes” a component, feature, structure, process, or characteristic B, it means that “A” is at least a partial cause of “B” but that there may also be at least one other component, feature, structure, process, or characteristic that assists in causing “B.” If the specification indicates that a component, feature, structure, process, or characteristic “may”, “might”, or “could” be included, that particular component, feature, structure, process, or characteristic is not required to be included. If the specification refers to “a” or “an” element, this does not mean there is only one of the described elements.

The foregoing descriptions are preferred embodiments of the present invention. As is understood by a person skilled in the art, the aforementioned preferred embodiments of the present invention are illustrative of the present invention rather than limiting the present invention. The present invention is intended to cover various modifications and similar arrangements included within the spirit and scope of the appended claims, the scope of which should be accorded the broadest interpretation so as to encompass all such modifications and similar structures.

What is claimed is:

1. A patterned magnetic thin film with 3D tube-shaped structure, comprising:
a substrate; and
at least one tube-shaped supportive layer rolled up onto said substrate, said tube-shaped supportive layer including a pattern portion having one or more magnetic material to attract an object into a hollow portion of said tube-shaped supportive layer.

2. The magnetic thin film of claim 1, wherein said substrate comprises a silicon material, said tube-shaped supportive layer comprises a photoresist and a SiO₂ for forming said pattern portion thereon by lithography.

3. The magnetic thin film of claim 1, wherein said magnetic material comprises a first layer, a second layer and a third layer.

4. The magnetic thin film of claim 3, wherein said first layer comprises Cr, Ti, Al as an adhesive layer, said second layer comprises Fe, Co, Ni and alloy thereof as a sensing layer, said third layer comprises Cr, Ti, Al or polymer as a protective layer.

5. The magnetic thin film of claim 1, wherein said pattern portion comprises a simple linear type or a wave-shaped type.

6. The magnetic thin film of claim 1, wherein said pattern portion further comprises a fishbone-shaped type.

7. The magnetic thin film of claim 1, wherein said pattern portion further comprises M periodic type, wherein said M is greater than 1.

8. The magnetic thin film of claim 5, wherein said pattern portion further comprises several elliptical type or oval type, being perpendicular to said simple linear type and said wave-shaped type to form a type with high magnetic anisotropy.

9. The magnetic thin film of claim 1, wherein a thermal expansion coefficient of said magnetic material is greater than that of said tube-shaped supportive layer, so as to bend towards a side of said magnetic material to roll upwards to away from said substrate.

10. The magnetic thin film of claim 1, wherein a thermal expansion coefficient of said magnetic material is smaller than that of said tube-shaped supportive layer, so as to bend towards a side of said tube-shaped supportive layer to roll downwards.

11. The magnetic thin film of claim 1, wherein a number of said objects is determined by the diameter and length of said tube-shaped supportive layer.

12. The magnetic thin film of claim 1, wherein a number of turn N of said tube-shaped supportive layer is greater than 1.

13. The magnetic thin film of claim 1, wherein a number of turn N of said tube-shaped supportive layer is equals to 1.
